(19) United States
(12) Patent Application Publication
(10) Pub. No.: US 2017/0354527 A1
(43) Pub. Date: Dec. 14, 2017

(54) Knee Brace Apparatus and Method

(71) Applicant: Todd Schwark, Kaslo (CA)

(72) Inventor: Todd Schwark, Kaslo (CA)

(21) Appl. No.: 15/178,840

(22) Filed: Jun. 10, 2016

(51) Int. Cl. A61F 5/01

(52) U.S. Cl. CPC A61F 5/0106

(57) ABSTRACT

The present invention is a system and method for providing knee support using a first restraining strap, constructed and arranged to wrap around the thigh of a user, a second restraining strap, constructed and arranged to wrap around the upper shin below the knee of a user, a first adjustable support positioned behind the thigh of a user, a second support positioned proximate to the calf leg portion of a user, wherein each of said first and second supports are formed with an internal cavity, being a first internal cavity and a second internal cavity, having a re-closable opening, wherein said internal cavity is filled with material to provide a particular amount of mass.

### BACKGROUND OF THE INVENTION

Knee injuries and knee supporting devices are quite common. However, one area that has yet to be addressed is placing a knee in a particular position of flexion without being unduly rigid and bothersome. The present invention provides for a system that controls the degree of flexion when a wearer is wearing a knee brace system according to the present invention and laying on a flat surface.

### SUMMARY OF THE INVENTION

This technology promotes the healing of overextended tendons and ligaments behind and around the knee as well as the hamstring area. The apparatus will be affixed one per leg around the knee area. It will have two pads behind the knee, one just above and one just below the back of the knee. They will be slightly wider than the knee itself and be about 3-4 inches thick. The other peripheral effects will be pain relief in the knee and the lower back as well as sleep comfort. The design allows for full range of sleep positions without any discomforts of any other similar inventions. There has been a strong trend in finding true health benefits and comfort with sleeping apparatus in the last few decades, but nothing seems all around beneficial as does this invention.

In one embodiment, the present invention is a system for providing knee support, said system comprising:

a first restraining strap, constructed and arranged to wrap around the thigh of a user;

a second restraining strap, constructed and arranged to wrap around the upper shin below the knee of a user;

a first adjustable support positioned behind the thigh of a user;

a second adjustable support positioned proximate to the calf leg portion of a user; wherein each of said first and second supports are formed with an internal cavity, being a first internal cavity and a second internal cavity, having a re-closable opening, wherein said internal cavity is filled with material to provide a particular amount of mass and impart a vertical height to support a users leg.

The material is selected from a solid, liquid, or gas. It is contemplated the internal material placed in the cavity be selected solely to impart a particular height or alternatively, the material is selected to provide vertical height and therapeutic effect, such as using a warm or hot solid, liquid or gas to provide heat therapy or a cold solid liquid or gas to provide cold therapy.

In one embodiment, each of said first and second straps are joined at a living hinge. This imparts a system that is connected and formed of one piece joined at the living hinge.

In one embodiment, each of said first and second internal cavity are filled with identical amounts of material to impart substantially congruent vertical support heights to provide support to a knee supported by the system.

In one embodiment, each of said first and second internal cavity are filled with different amounts of material to impart different vertical support heights to provide support to a knee supported by the system.

In one embodiment, each of said first and second internal cavity are filled with an amount of material to impart a vertical support height to provide support to a knee supported by the system and position said knee in a fixed position defined by an angle formed by thigh and shin position and the knee forming an angular vertex. In one embodiment, the angle is between about 135° and 175° (degrees). In one embodiment, the angle is greater than 135° and less than 175°.

In one embodiment, the distance between each of said first and second support are independently adjustable along a lower portion each of said first and second straps.

In one embodiment, the distance between each of said first and second support is independently adjustable along a lower portion each of said first and second straps and each of said each of said first and second internal cavity are independently adjustable in the amount of material positioned therein.

Also contemplated is a method of stabilizing and immobilizing a knee, said method comprising the steps of:
providing a system according to the present invention; securing said system to the leg of a user using each of said straps;
providing a mass of material to each of said cavities;
placing a patient wearing said system on a substantially firm surface to immobilize the knee in a fixed angular orientation.

This method also includes determining a desired height for each of the first and second cavities and filling the cavities with desired amounts of materials to impart the desired vertical height for each. The vertical height desired will be directly related to the desired angular offset between shin and thigh.

The method also includes the steps of measuring the desired distance between each of the first and second supports and positioning each support at a distance required by said measuring.

The method also includes the step of imparting a flexible range of motion when wearing said system, said range of motion permitting a change in the angular offset and allowing a wearer flexibility to move their leg when wearing the system in order to stand, walk, and turn in bed.

The system of the preset invention represents an improvement at least in one embodiment because it holds a knee in place in most positions and follows the body during sleep. No other product or system allows for full range of movement without any adjustments. The system also stays on if leaving the bed so as to allow freedom of assistance in a serious condition such as a hospital for example, going to lavatory, changing a channel, adjusting heat, opening window, and return to bed. This is an advancement as conventional knee supports are rigid and restrict movement. The present invention provides for positioning and maintaining the knee of a wearer in a desired elevated position and desired angle, while providing a flexible device that permits movement.

In one embodiment, by providing a system in which each of the elevation height and distance between supports are independently adjustable, the present invention represents a significant improvement in that one size will fit many patients of many sizes which is contrary to current systems that must be stocked and administered in a range of sizes to match the physical stature of a patient.

### BRIEF DESCRIPTION OF THE SEVERAL VIEWS OF THE DRAWING

FIG. 1 is a perspective view of the present invention in an environment of use.

FIG. 2 is a cross-section along section lines A-A from FIG. 1.

FIG. 2A is a cross-section along section lines A-A from FIG. 1 in an embodiment with an internal bladder.

FIG. 3 is a plan view of the underside of the present invention.

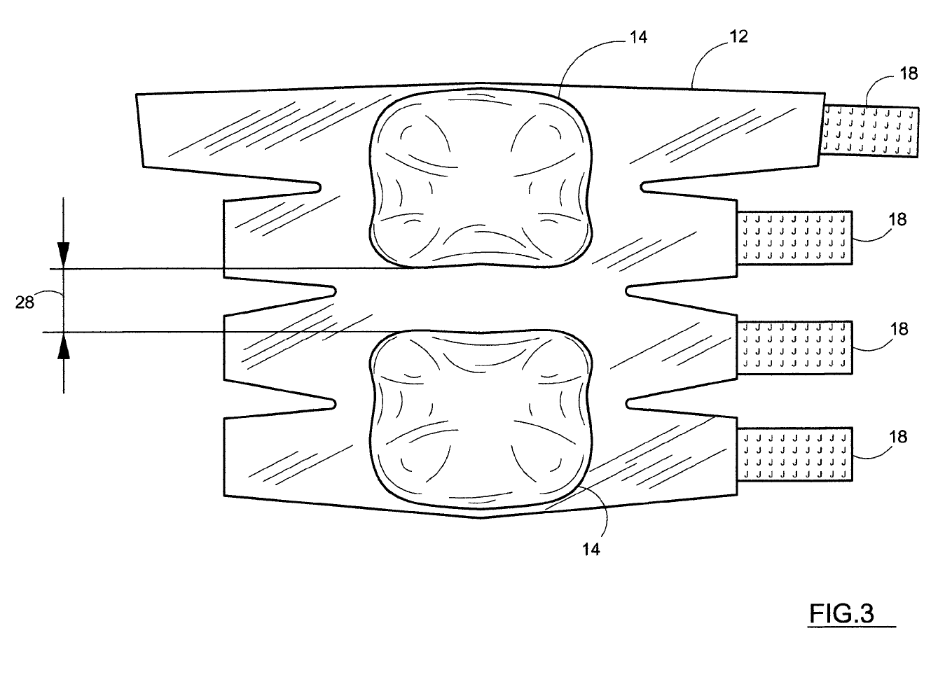

FIG. 4 is a top plan view of one embodiment of the present invention.

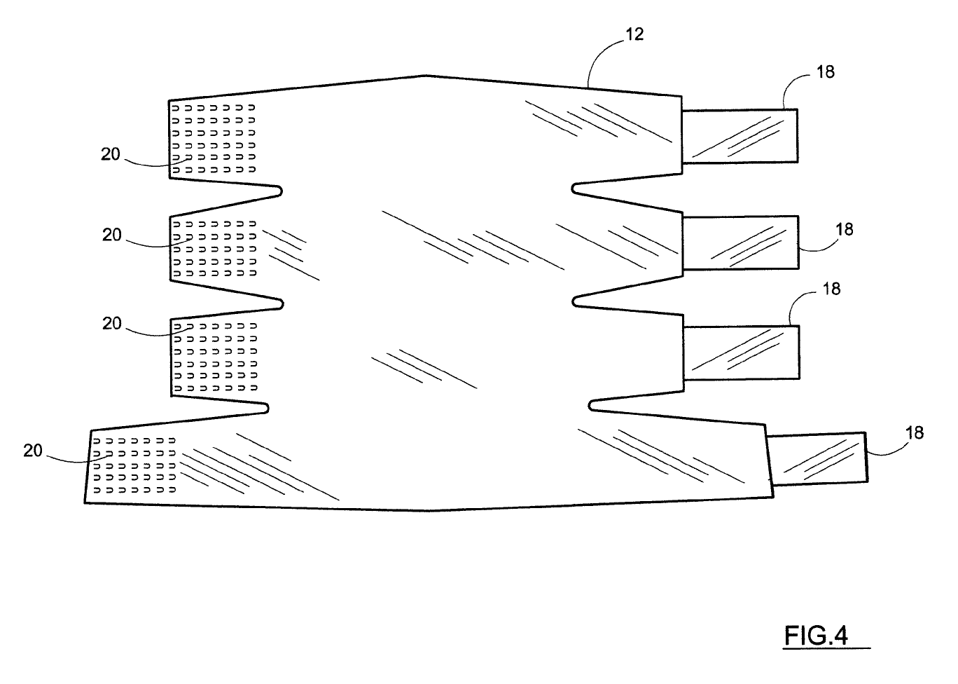

FIG. 5 is a side perspective view of a multiple joint elevation brace according to one embodiment of the present invention.

### DETAILED DESCRIPTION OF THE PREFERRED EMBODIMENT

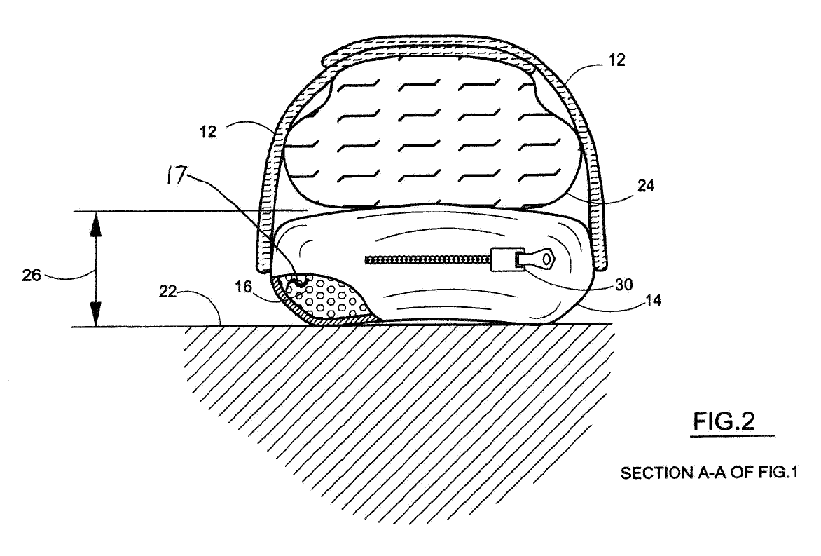

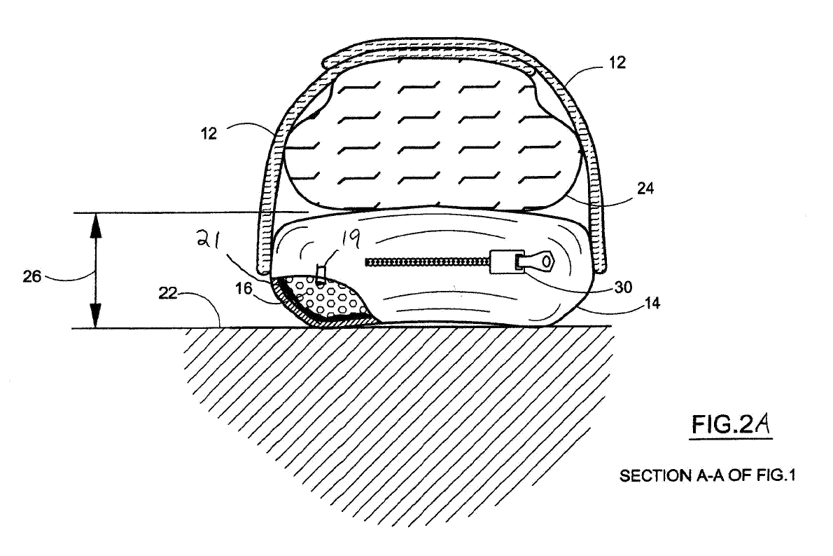

As generally understood, knee brace system 10 includes an upper restraining strap 12, constructed and arranged to attach about the thigh of a user. One or more supports 14 are positioned under the thigh and/or shin regions as needed in order to provide the desired elevation of knee 24 in system 10. Support 14, in one embodiment, has an accessible opening 30, demonstrated in FIG. 2 as a zipper, although any re-closable opening is contemplated, whereby filler material 16 is inserted into the interior cavity 17 of support 14. In this arrangement, the amount of bulk is regulated and customizable to provide the desired amount of elevation when wearing system 10 of the present invention. In one embodiment, filler material 16 is solid and selected fro material such as cotton, foam, polyfibers and similar type cushioning material.

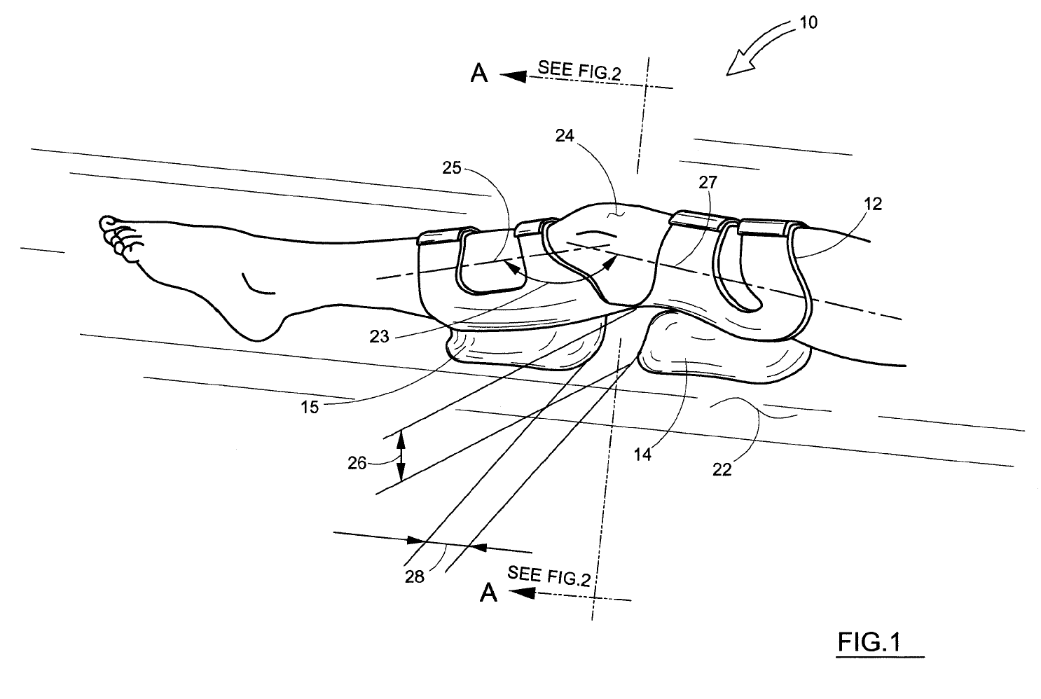

It is contemplated that system 10 is attached using hook fasteners 18 that mate with loop fasteners 20 in a common hook-and-loop fastening relationship as is commonly known. Dimension 26 relates to the vertical displacement height that the back part of knee 24 is elevated off of resting plane 22, on which supports 14 are resting. Dimension 28 refers to the distance between the end of a first support 14 and second support 15 as demonstrated in FIG. 1. In one embodiment, each of dimension 26 and dimension 28 are configured as being adjustable in order to provide a customized fit for a patient.

As generally understood, a person with a knee injury typically requires the knee being in a fixed and somewhat flexed position. However, current knee braces are deficient in that they do not provide for relative degrees of flexion. The present invention addresses this by providing adjustable supports 14 that will position knee 24 in a desired degree of flexion and hold knee 24 in that position for prolonged periods of time. It is contemplated that the present invention will be a significant advancement in that the position of the knee is more closely controlled than typical braces provide.

As demonstrated in FIG. 1, angle offset 23 is the angle formed by the thigh as one segment 27, the shin as a second segment 25 and the knee as the vertex. Because different conditions have different requirements, the present invention provides an adjustable positioning system and an angular offset determined by a medical practitioner of between about 135-175 degrees.

In one embodiment, each cavity 17 has a bladder 21 that is operatively associated with a two-way fill and empty valve 19. Air or liquid can be adjusted in the bladder 21 in order to impart a desired angular offset.

In order to achieve a desired vertical offset for each of first support 14 and second support 15, the inner filling material 16 can be varied in any combination such as:

The material in said first adjustable support is a solid and material in said second adjustable support is a solid.

The material in said first adjustable support is a solid and material in said second adjustable support is a liquid.

The material in said first adjustable support is a solid and material in said second adjustable support is a gas.

The material in said first adjustable support is a liquid and material in said second adjustable support is a solid.

The material in said first adjustable support is a liquid and material in said second adjustable support is a liquid.

The material in said first adjustable support is a liquid and material in said second adjustable support is a gas.

The material in said first adjustable support is a gas and material in said second adjustable support is a solid.

The material in said first adjustable support is a gas and material in said second adjustable support is a liquid.

The material in said first adjustable support is a gas and material in said second adjustable support is a gas.

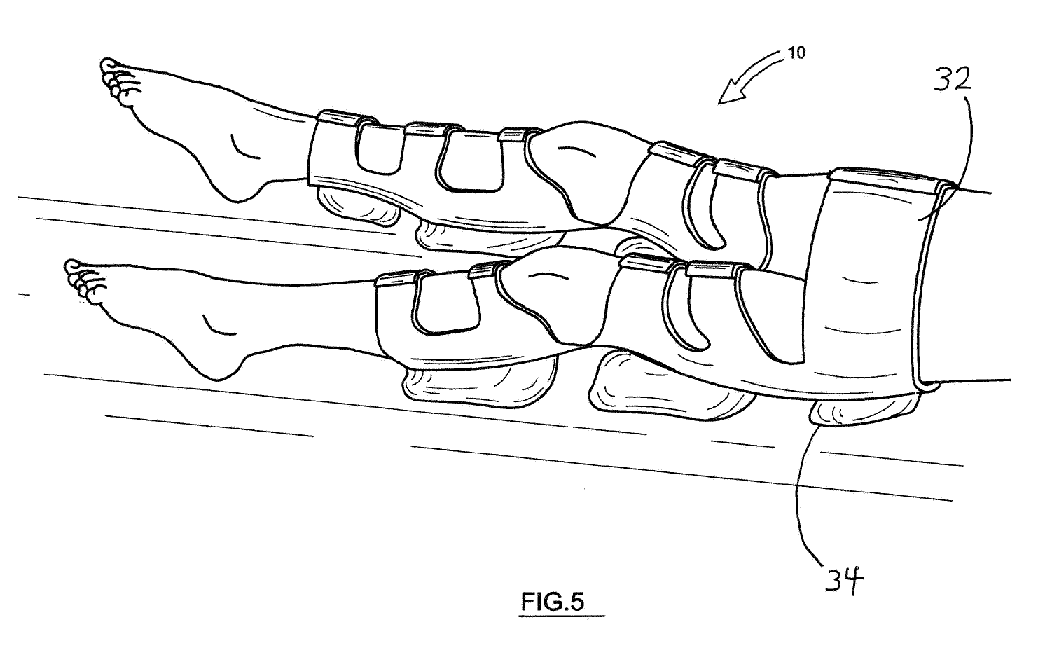

In one embodiment, as shown in FIG. 5, the invention includes waist band 32 that is attached to system 10 and wraps around the waist of the user. Optionally, a third support 34 constructed and arranged similar to first support 14 and second support 15 is positioned under waistband 32. Waistband 32 is secured similarly by adjustable and removable mechanisms including, but not limited to, hook and loop fasteners. In the embodiment utilizing waistband 32, the system provides even greater ability to move while wearing the system. This is a significant improvement over current knee braces that are rigid and significantly immobilize the knee and also the user. The present invention supports the knee while lying substantially horizontal and allows a user freedom of motion when desired in situations relating to standing and walking.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed is:

1. A system for providing knee support, said system comprising: a first restraining strap, constructed and arranged to wrap around the thigh of a user;
a second restraining strap, constructed and arranged to wrap around the upper shin below the knee of a user;
a first adjustable support positioned behind the thigh of a user;
a second adjustable support positioned proximate to the calf leg portion of a user; wherein each of said first and second supports are formed with an internal cavity, being a first internal cavity and a second internal cavity, having a re-closable opening, wherein each of said internal cavities is filled with material to provide a particular amount of mass and impart a vertical height to support a users leg.

2. The system of claim 1 wherein said material is selected from a solid, liquid, or gas.

3. The system of claim 1 wherein material in said first adjustable support is a solid and material in said second adjustable support is a solid.

4. The system of claim 1 wherein material in said first adjustable support is a solid and material in said second adjustable support is a liquid.

5. The system of claim 1 wherein material in said first adjustable support is a solid and material in said second adjustable support is a gas.

6. The system of claim 1 wherein material in said first adjustable support is a liquid and material in said second adjustable support is a solid.

7. The system of claim 1 wherein material in said first adjustable support is a liquid and material in said second adjustable support is a liquid.

8. The system of claim 1 wherein material in said first adjustable support is a liquid and material in said second adjustable support is a gas.

9. The system of claim 1 wherein material in said first adjustable support is a gas and material in said second adjustable support is a solid.

10. The system of claim 1 wherein material in said first adjustable support is a gas and material in said second adjustable support is a liquid.

11. The system of claim 1 wherein material in said first adjustable support is a gas and material in said second adjustable support is a gas.

12. The system of claim 1 where in each of said first and second straps are joined at a living hinge.

13. The system of claim 1 wherein each of said first and second internal cavity are filled with identical amounts of material to impart a vertical support height to provide support to a knee supported by the system.

14. The system of claim 1 wherein each of said first and second internal cavity are filled with different amounts of material to impart a vertical support height to provide support to a knee supported by the system.

15. The system of claim 1 wherein each of said first and second internal cavity are filled with an amount of material to impart a vertical support height to provide support to a knee supported by the system and position said knee in a fixed position defined by an angle formed by thigh and shin position and the knee forming an angular vertex.

16. The system of claim 1 wherein each of said first and second internal cavity are filled with a mass of material to provide support to a knee supported by the system and position said knee in a fixed position defined by an angle formed by thigh and shin position and the knee forming an angular vertex, said angle being between about 135 and 175 degrees.

17. The system of claim 1 wherein a distance between each of said first and second support are independently adjustable along a lower portion each of said first and second straps.

18. The system of claim 1 wherein a distance between each of said first and second support is independently adjustable along a lower portion each of said first and second straps and each of said each of said first and second internal cavity are independently adjustable in the amount of material positioned therein.

19. A method of stabilizing and immobilizing a knee, said method comprising the steps of:
providing a system according to claim 1;
securing said system to the leg of a user using each of said straps;
providing a mass of material to each of said cavities;
placing a patient wearing said system on a substantially firm surface to immobilize the knee in a fixed angular orientation.

20. The method of claim 19 further comprising the step of imparting a flexible range of motion when wearing said system, said range of motion permitting a change in the angular offset and allowing a wearer flexibility to move their leg when wearing the system in order to stand, walk, and turn in bed.
